(19) United States
(12) Patent Application Publication
(10) Pub. No.: US 2014/0102458 A1
(43) Pub. Date: Apr. 17, 2014

(54) TUBE SECUREMENT DEVICE

(71) Applicants: Tabitha Landow, Bloomfield, CT (US); Yvonne Szacki, Brooklyn, NY (US)

(72) Inventors: Tabitha Landow, Bloomfield, CT (US); Yvonne Szacki, Brooklyn, NY (US)

(21) Appl. No.: 13/980,341

(22) PCT Filed: Jan. 18, 2012
(86) PCT No.: PCT/US2012/021668

§ 371 Date: Jan. 6, 2014

Related U.S. Application Data
(60) Provisional application No. 61/433,945, filed on Jan. 18, 2011.
(60) Provisional application No. 61/528,871, filed on Aug. 30, 2011.

(51) Int. Cl. A61D 7/04; A61M 16/04; A61M 16/06

(52) U.S. Cl. CPC A61D 7/04; A61M 16/0683; A61M 16/0465
USPC 128/207.11; 128/207.17

(57) ABSTRACT

A securement device used to secure an anesthesia mask or an endotracheal tube to an animal patient during surgery, includes an elongated flexible element having two ends. A first slidable connector is threaded through the two ends and is movable along the elongated flexible element. The first connector creates a first loop in the elongated flexible element such that the first loop is wrappable around the anesthesia mask or the endotracheal tube. The first connector is slidable for securing the first loop around the anesthesia mask or endotracheal tube. A second slidable connector is threaded through the two ends, and creates a second loop in the elongated flexible element. The second connector is positioned such that the second loop is securely attachable to a head of the animal patient using the second connector sliding along the elongated flexible element.

### CROSS-REFERENCE TO RELATED APPLICATION

The present application claims priority under 35 U.S.C. §119 of U.S. Provisional Application Ser. No. 61/433,945, filed Jan. 18, 2011, entitled ENDOTRACHEAL TUBE TIE, and U.S. Provisional Application Ser. No. 61/528,871, filed Aug. 30, 2011, entitled TUBE SECUREMENT DEVICE, the disclosures of which are hereby incorporated herein by reference in their entirety for all that they disclose and teach.

### BACKGROUND OF THE INVENTION

Occasionally, animals such as cats and dogs will have to undergo surgery. During surgery, it is often desirable to have an endotracheal tube inserted into the animal, or an anesthesia mask attached to the animal. Endotracheal tubes and anesthesia masks provide important functions during surgery.

### SUMMARY OF THE INVENTION

A securement device used to secure an anesthesia mask to an animal patient during surgery includes an elongated flexible element having two ends. A first slidable connector is threaded through the two ends. The first connector is movable along the elongated flexible element and the first connector creates a first loop in the elongated flexible element such that the first loop is wrappable around the anesthesia mask. The first connector is slidable for securing the first loop around the anesthesia mask. A second slidable connector is threaded through the two ends. The second connector is movable along the elongated flexible element. The second connector creates a second loop in the elongated flexible element, and the second connector is positioned to create an opening between the first connector and the second connector such that the second loop is securely attachable to a head of the animal patient using the second connector sliding along the elongated flexible element to a tightening position.

A securement device used to secure an endotracheal tube to an animal patient during surgery includes an elongated flexible element having two ends. A first slidable connector is threaded through the two ends. The first connector is movable along the elongated flexible element and the first connector creates a first loop in the elongated flexible element such that the first loop is wrappable around the endotracheal tube. The first connector is slidable for securing the first loop around the endotracheal tube. A second slidable connector is threaded through the two ends. The second connector is movable along the elongated flexible element. The second connector is positioned to creates an opening between the first connector and the second connector such that the second loop is securely attachable to a portion of the animal patient head by using the second connector sliding along the elongated flexible element in a tightening position.

A method of maintaining a medical device in a secure position on an animal patient during surgery using a securement device which includes: sliding a first connector through a first end and second end of an elongated flexible element; sliding the first connector to form a loop in the elongated flexible element, the loop being wrappable around a medical device; tightening the first connector such that the loop is tightly secure around the medical device; sliding a second connector through the first end and the second end of the elongated flexible element so that an opening is created in the elongated flexible element between the first connector and the second connector; attaching the opening to a head of the animal patient by inserting the head in the opening and sliding the second connector to a tightening position.

### BRIEF DESCRIPTION OF THE DRAWINGS

These and other objects, features and advantages of the present invention will become apparent from the following detailed description of illustrative embodiments thereof, which is to be read in connection with the accompanying drawings, in which:

FIG. 1 is an isometric side view of tube securement device securing an anesthesia mask;

FIG. 2 is an isometric view of tube securement device;

FIG. 3A is an isometric top view of tube securement device securing on the front of the mask of FIG. 1;

FIG. 3B is an isometric view of a tube securement device secured to the front of the mask of FIG. 3A;

FIG. 4A is an isometric side view of a tube securement device securing an anesthesia mask of FIG. 1;

FIG. 4B is a top isometric view of FIG. 4A;

FIG. 5A is a side isometric view of a tube securement device connecting to an endotracheal tube;

FIG. 5B is a side isometric view of a tube securement device secured to the endotracheal tube of FIG. 5A;

FIG. 6A is an isometric side view of a tube securement device secured to a nose of an animal;

FIG. 6B is an isometric side view of a tube securement device secured to a jaw of an animal; and

FIG. 7 is an isometric side view of a tube securement device secured to a neck of an animal.

### DETAILED DESCRIPTION OF THE INVENTION

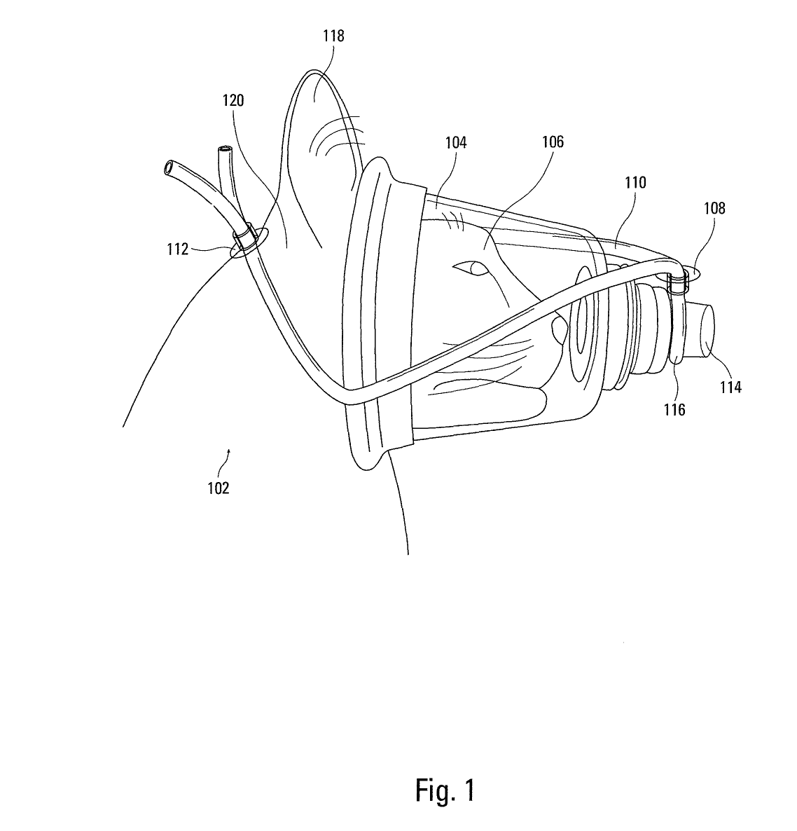

Referring to FIG. 1, in one embodiment of the invention, an animal is shown wearing an anesthesia mask. During surgery, it can be difficult to secure an anesthesia mask on an animal. Oftentimes, rolled gauze is used, where the rolled gauze can be easily shifted and can be difficult to quickly untie in case of an emergency. Anesthesia masks, such as anesthesia mask 104, may take various forms and have various sizes, which do not lend themselves to a simple way of securing the mask to the animal patent. As such, it may be difficult to properly secure a mask to the animal patient because of the different head sizes and shapes of the animal patient and the various types of masks that are used. Further, movement of the animal, either before or after the animal is anesthetized, may cause dislodgement and interruption of the surgery procedure, if the mask is not properly secured to the animal patient and becomes dislodged.

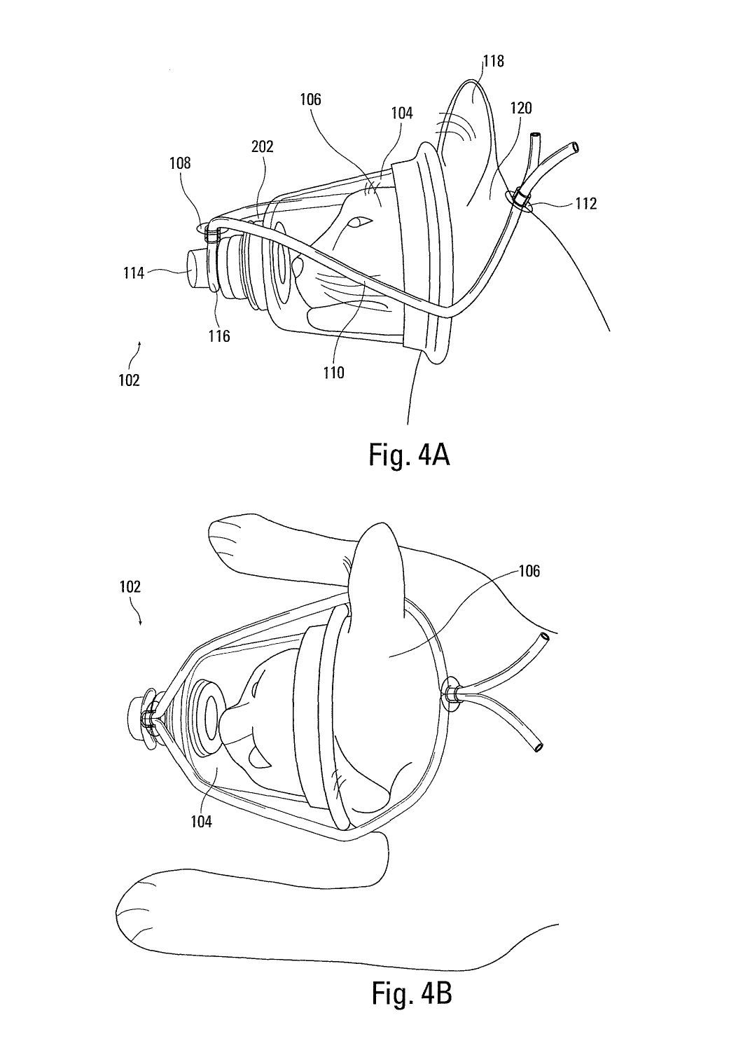

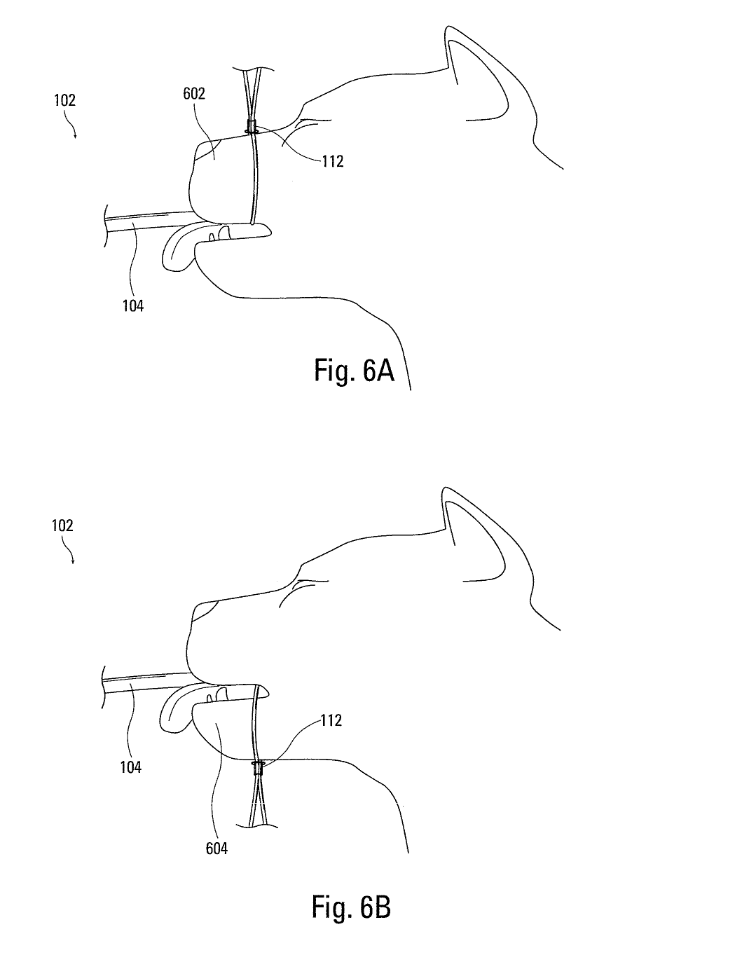

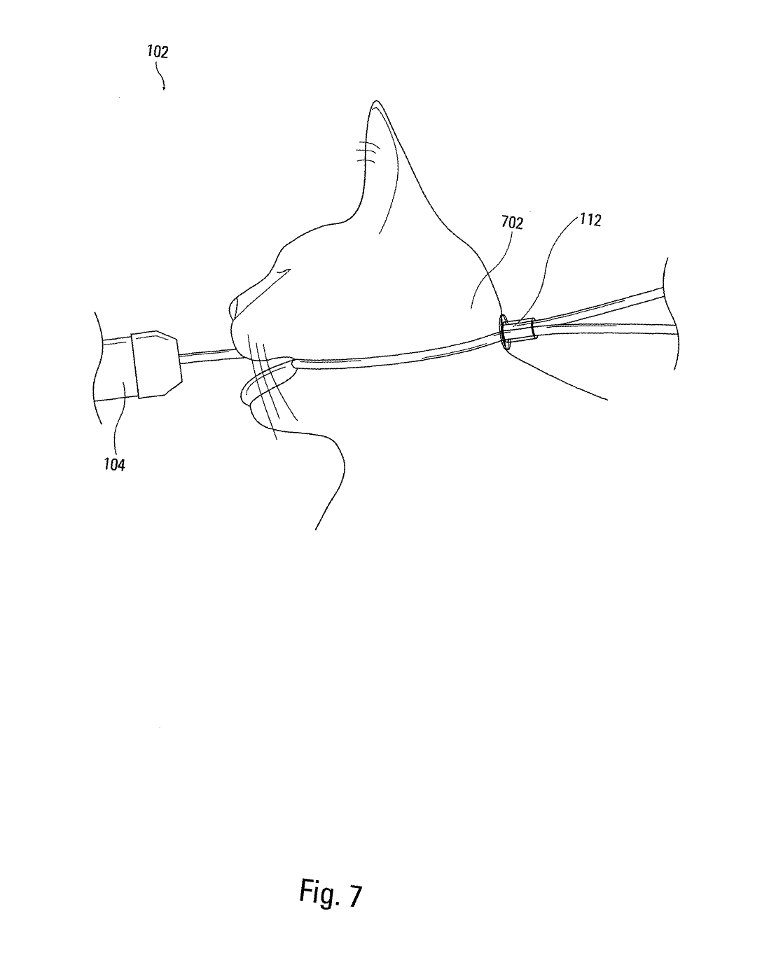

Providing a securement device that can work with various animals having different sizes of heads and different shaped heads and mouths, in a manner which properly secures the mask to the animal is beneficial. The ability to use a securement device to also secure endotracheal tubes to various animal species is even more beneficial. A securement device 102 can be used to secure anesthesia mask 104 to the head 106 of an animal patient, as illustrated in FIGS. 1, 4A and 4B, as well as securing an endotracheal tube to the head of an animal patient, as illustrated in FIGS. 6A, 6B and 7. When using the securement device 102 to secure an anesthesia mask 104, the anesthesia mask 104 (FIG. 1) can be quickly secured and removed before and after surgery so that anesthesia mask 104 does not easily shift.

Referring again to FIG. 1, a loop 116 of tubing 110 is wrapped around front 114 and tightened by a first sliding connector 108. Tubing 110 is then wrapped behind the animal's head 106 and tightened by a second sliding connector 112 along tubing 110 so that tubing 110 is secured behind the ears 118 and around the neck 120 of the animal. In other words, when an animal is brought in for surgery, a first loop 116 wraps around front 114 and is tightened around front 114 by tightening sliding connector 108 to secure loop 116 around front 114. The anesthesia mask 104 is then placed on animal head 106 so that tubing 110 is wrapped around animal head 106 by tightening sliding connector 112 in a secure fashion so that a second loop 202 of the tube securement device is in place behind ears 118 and around neck 120, so the animal is unable to shift the tube securement device 102. Sliding connectors 108, 112 are able to easily slide along tubing 110 to allow easy and rapid attachment, as well as detachment in the case of an emergency, and allows ease of use.

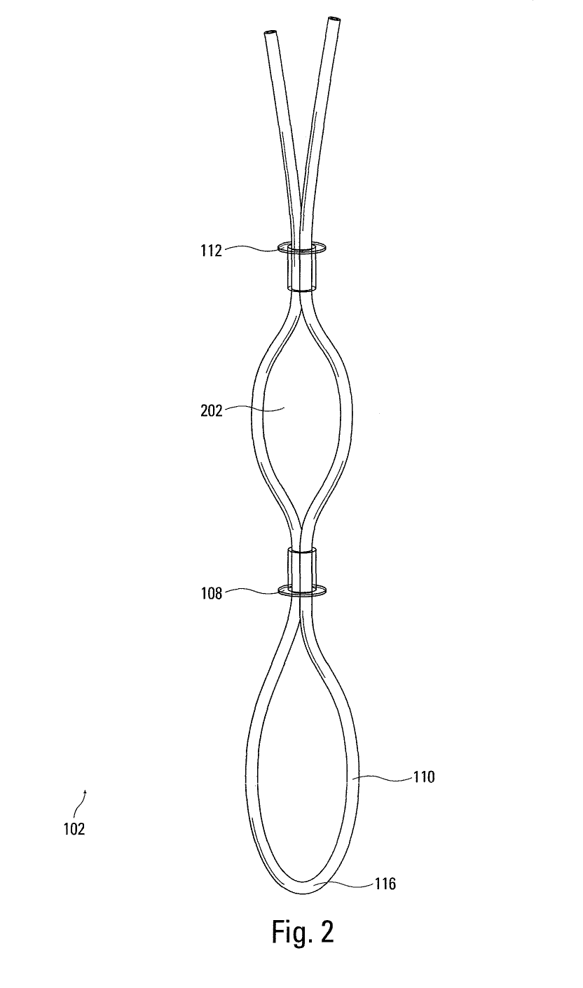

FIG. 2 is an isometric side view of the tube securement device 102. Tubing 110 may be made of a thin and strong plastic tubing that is able to secure challenging animal patients, such as pugs and felines. Although the embodiment of the invention shown in Figures uses plastic tubing, other embodiments of an elongated flexible element may be used. For example, flexible straps may be used, which may also be resilient and which may include elastic properties or materials. Further, the connectors may include buckles, straps, clips or other slidable connectors or slidable fastening devices, as well as adjustable connectors or removable connectors. In an alternative embodiment, an elongated element may be used, for example, a strap which may be rigid, and be comprised of a soft and strong material, such as a thermoplastic, for example Nylon.

The tube securement device is lightweight, inexpensive, reusable, durable and can hang in an induction area readily available for use. The tube securement device 102 is also a one size fits all device, so various sizes do not have to be ordered. Tube securement device 102 comprises tubing 110 that creates loop 116 by sliding connector 108. Sliding connector 108 is capable of sliding up and down tubing 110 in an easy manner. Sliding connector 112 creates loop 202 by securing tubing 110 in a separate location. Sliding connectors 108, 112 create loop 116 and loop 202. Sliding connectors 108, 112 are easy to slide up and down tubing 110, which facilitates in the securement of tube securement device 102 during surgery, but retain sufficient friction to prevent movement by the animal patient.

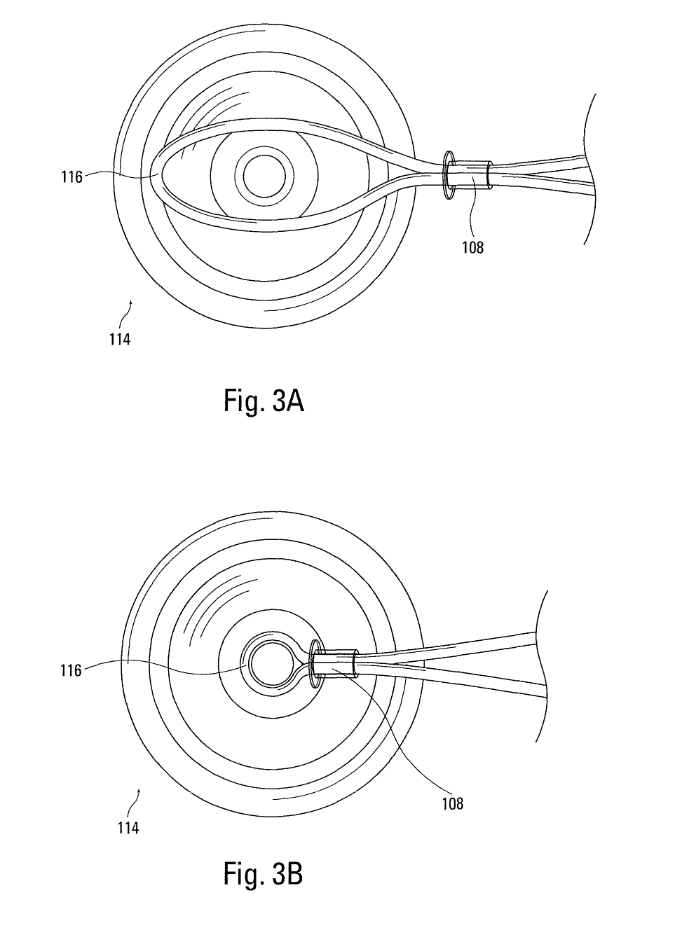

FIG. 3A is a front isometric view of anesthesia mask 104. FIG. 3A shows loop 116 being wrapped around front 114, and sliding connector 108 is in a non-tightened position, so that loop 116 is easily wrapped around front 114. FIG. 3B shows loop 116 being tightly wrapped around front 114 by sliding connector 108 being tightened, in order to secure loop 116 around front 114.

FIG. 4A is an isometric side view of a tube securement device being used with an anesthesia mask 104. Loop 116 is secured around front 114 of anesthesia mask 104 by sliding connector 108 in a secure fashion to front 114. Loop 202 is then wrapped around animal head 106, behind ears 118, and around neck 120 by tightening sliding connector 112 along tubing 110, FIG. 4B is a top view of FIG. 4A showing tube securement device 102 so that the anesthesia mask 104 is secure against animal head 106.

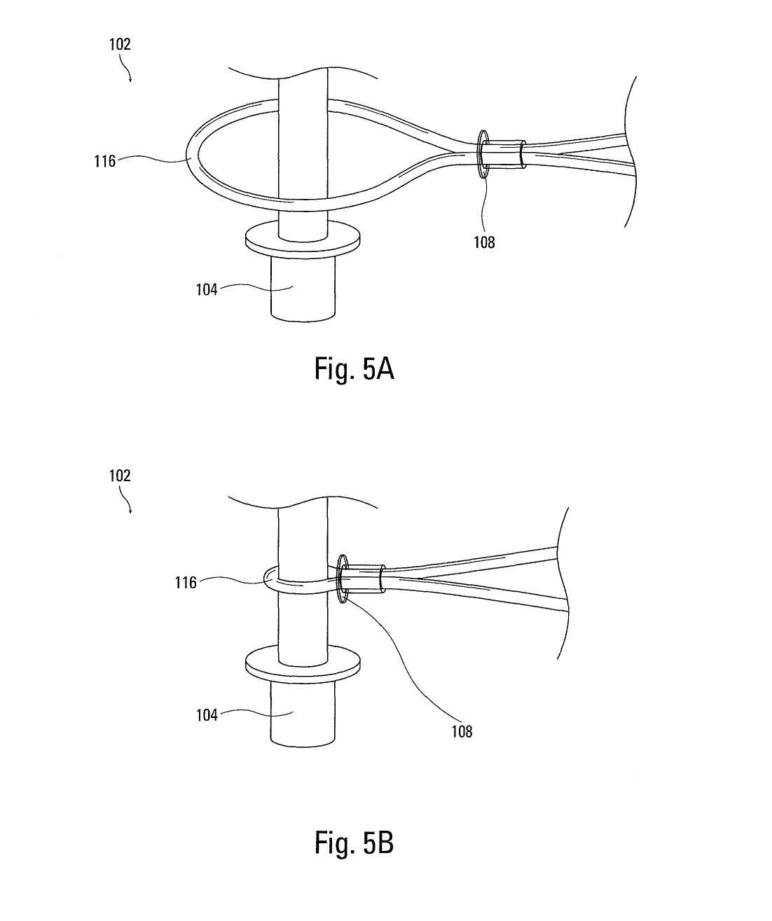

Tube securement device 102 can also be used to secure an endotracheal tube in an animal patient's mouth during surgery. FIGS. 5A and 5B are side views of tube securement device 102. Endotracheal tubes are breathing tubes used during surgery that enable a patient to breath freely during surgery. If an endotracheal tube shifts or becomes dislodged, a patient can choke or have other complications. FIG. 5A shows loop 116 of tube securement device 102 wrapped around endotracheal tube 104. Loop 116 is easily wrapped around endotracheal tube 104 by having sliding connector 108 in a loosened position. FIG. 5B shows loop 116 wrapped securely around endotracheal tube 104 by tightening sliding connector 108 in a secure position around endotracheal tube 104.

Referring to FIGS. 6A, 6B and 7, three different positions of the tube securement device are shown. FIG. 6A shows tube securement device 102 connected to endotracheal tube 104 that is inserted in the patient's mouth. Tube securement device 102 is then slid over nose 602 by sliding connector 112 against nose 602 on the upper jaw of the animal patient to securely hold endotracheal tube 104 in place. FIG. 6B shows endotracheal tube 104 inserted in the patient's mouth, with sliding connector 112 of tube securement device 102 fitting securely around the lower jaw 604 of the patient. FIG. 7 shows tube securement device 102 being secured along patient's neck 702, by tube securement device 102 being connected to endotracheal tube 104 and sliding connector 112 being securely connected around neck 702 of the patient.

Hence, the tube securement device can be used on various species of animal patients that have different size heads and differently shaped heads. The tube securement device is capable of securely attaching an anesthesia mask to the various species of animal patients. In addition, the tube securement device 102 can be used to secure endotracheal tubes to various species of animals. The tube securement device is lightweight, inexpensive, re-useable, durable and one size fits various species of animals and various shapes and sizes of the jaws and heads of these vanous species.

The foregoing description of the invention has been presented for purposes of illustration and description. It is not intended to be exhaustive or to limit the invention to the precise form disclosed, and other modifications and variations may be possible in light of the above teachings. The embodiment was chosen and described in order to best explain the principles of the invention and its practical application, to thereby enable others skilled in the art to best utilize the invention in various embodiments and various modifications as are suited to the particular use contemplated. It is intended that the appended claims be construed to include other alternative embodiments of the invention.

What is claimed is:

1. A securement device used to secure an anesthesia mask to an animal patient during surgery, comprising:
an elongated flexible element having two ends;
a first slidable connector threaded through said two ends, said first connector being movable along said elongated flexible element and said first connector creates a first loop in said elongated flexible element such that said first loop is wrappable around said anesthesia mask and said first connector is slidable for securing said first loop around said anesthesia mask;
a second slidable connector threaded through said two ends, said second connector being movable along said elongated flexible element, said second connector creates a second loop in said elongated flexible element, and said second connector being positioned to create an opening between said first connector and said second connector such that said second loop is securely attachable to a head of said animal patient using said second connector sliding along said elongated flexible element to a tightening position.

2. The securement device of claim 1, wherein said opening is secured behind the ears of said animal patient by sliding said second connector in the tightening position behind said ears on said head.

3. The securement device of claim 1, wherein the elongated flexible element is a plastic tube.

4. A securement device used to secure an endotracheal tube to an animal patient during surgery, comprising:
an elongated flexible element having two ends;
a first slidable connector threaded through said two ends, said first connector being movable along said elongated flexible element and said first connector creates a first loop in said elongated flexible element such that said first loop is wrappable around said endotracheal tube and said first connector is slidable for secure said first loop around said endotracheal tube;
a second slidable connector threaded through said two ends, said second connector being movable along said elongated flexible element, and said second connector being positioned to creates an opening between said first connector and said second connector such that said second loop is securely attachable to a portion of said animal patient head by using said second connector sliding along said elongated flexible element into a tightening position.

5. The securement device of claim 4, wherein said opening is secured to said head portion of said animal patient by sliding said second connector in a tightening position around a back portion of a neck of said animal patient.

6. The securement device of claim 4, wherein said opening is secured around said head portion by sliding said second connector in the tightening position along said nose on an upper jaw portion of said animal patient.

7. The securement device of claim 4, wherein said opening is secured around said head portion by sliding said second connector in a tightening position along a lower jaw portion of said animal patient.

8. The securement device of claim 4, wherein the elongated flexible element is a plastic tube.

9. A method of maintaining a medical device in a secure position on an animal patient during surgery using a securement device, comprising:
sliding a first connector through a first end and second end of an elongated flexible element;
sliding said first connector to form a loop in the elongated flexible element, said loop being wrappable around a medical device;
tightening said first connector such that said loop is tightly secure around said medical device;
sliding a second connector through said first end and said second end of said elongated flexible element so that an opening is created in said elongated flexible element between said first connector and said second connector;
attaching said opening to a head of said animal patient by inserting said head in said opening and sliding said second connector to a tightening position.

10. The method of claim 9, wherein said opening is secured behind ears on said head of said animal patient by sliding said second connector in the tightening position.

11. The method of claim 9, wherein said opening is secured around an upper jaw portion on said head of said animal patient by sliding said second connector in a second tightening position.

12. The method of claim 9, wherein said opening is secured around a lower jaw portion on said head by sliding said second connector in a third tightening position around said lower jaw portion.

13. The method of claim 9, wherein the elongated flexible element is a plastic tube.
